(19) United States
(12) Patent Application Publication
(10) Pub. No.: US 2012/0201798 A1
(43) Pub. Date: Aug. 9, 2012

(54) COMPOSITIONS AND METHODS AND USES RELATED THERETO

(76) Inventors: Riina Kekkonen, Helsinki (FI); Minja Miettinen, Helsinki (FI)

(21) Appl. No.: 13/501,547

(22) PCT Filed: Oct. 13, 2010
(86) PCT No.: PCT/FI10/50792

§ 371 Date: Apr. 12, 2012

(30) Foreign Application Priority Data
Oct. 13, 2009 (FI) 20096058

(51) Int. Cl. A61K 35/74; A61P 31/16; A61P 31/14; C12N 1/20; A61P 31/12

(52) U.S. Cl. 424/93.45; 435/252.9

(57) ABSTRACT

The present invention relates to the fields of life sciences and food, feed or pharmaceutical industry. Specifically, the invention relates to a composition comprising probiotics consisting of Lactobacillus rhamnosus LC70 alone or Lactobacillus rhamnosus GG and Lactobacillus rhamnosus LC705. Also the invention relates to the composition for use as a medicament. Furthermore, the present invention relates to uses of Lactobacillus rhamnosus LC705 alone or together with Lactobacillus rhamnosus GG for the manufacture of a composition for the treatment and/or prevention of a respiratory infection and for intensifying resistance against viruses causing respiratory infections in a subject. Furthermore, the present invention describes uses of Lactobacillus rhamnosus GG for the manufacture of a composition for the treatment and/or prevention of a respiratory infection in an adult and for intensifying resistance against viruses causing respiratory infections in an adult subject. Still, the present invention relates to uses of Lactobacillus rhamnosus for the manufacture of a composition for reducing, delaying or inhibiting influenza virus replication and for increasing antiviral cytokine(s) in a subject to be or being infected with a respiratory infection. Still, the present invention relates to methods of treating or preventing a respiratory infection in a subject or in an adult subject, intensifying resistance against viruses causing respiratory infections in a subject or in an adult subject, reducing, delaying or inhibiting influenza virus replication in a subject and increasing antiviral cytokine(s) in a subject to be or being infected with a respiratory infection.

### FIELD OF THE INVENTION

The present invention relates to the fields of life sciences and food, feed or pharmaceutical industry. Specifically, the invention relates to a composition comprising probiotics consisting of Lactobacillus rhamnosus LC705 alone or Lactobacillus rhamnosus GG and Lactobacillus rhamnosus LC705. Also the invention relates to the composition for use as a medicament. Furthermore, the present invention relates to uses of Lactobacillus rhamnosus LC705 alone or together with Lactobacillus rhamnosus GG for the manufacture of a composition for the treatment and/or prevention of a respiratory infection and for intensifying resistance against viruses causing respiratory infections in a subject. Furthermore, the present invention describes uses of Lactobacillus rhamnosus GG for the manufacture of a composition for the treatment and/or prevention of a respiratory infection in an adult and for intensifying resistance against viruses causing respiratory infections in an adult subject. Still, the present invention relates to uses of Lactobacillus rhamnosus for the manufacture of a composition for reducing, delaying or inhibiting influenza virus replication and for increasing antiviral cytokine(s) in a subject to be or being infected with a respiratory infection. Still, the present invention relates to methods of treating or preventing a respiratory infection in a subject or in an adult subject, intensifying resistance against viruses causing respiratory infections in a subject or in an adult subject, reducing, delaying or inhibiting influenza virus replication in a subject and increasing antiviral cytokine(s) in a subject to be or being infected with a respiratory infection.

### BACKGROUND OF THE INVENTION

Probiotics have been used for the prevention and treatment of a diverse range of disorders such as arterial hypertension, vascular diseases, allergies, cancer, atopic diseases, viral or infectious diseases, dental caries, IBS, IBD, mucosal inflammation, gut permeability disorders, obesity, metabolic syndrome, oxidative stress and abdominal pain.

Based on early diarrheal studies, probiotics are known to reduce infections in the gastrointestinal tract. The present scientific data supports the assumption that the effects of probiotics on well-being as well as prevention and treatment of diseases is based on the ability to modify the microbiota in the gastrointestinal tract and to displace the pathogens. However, now there is increasing evidence that specific probiotics may also reduce infections outside the gastrointestinal tract.

Mucosal epithelial surfaces in the mouth and gastrointestinal and respiratory tracts constantly fight against infectious agents such as viruses. The commensal microbiota on these surfaces protects the body against pathogenic organisms by metabolic and regulatory substances and by competing for nutrients and available adhesion sites on the mucosa. Microbiota develops and changes during the life time, but normally in a healthy subject it contains a diversity of bacterial species.

Acute respiratory infections affecting the upper or lower respiratory tract are the most common health problems among children and the elderly, though the incidence is high in all age groups. These respiratory infections cause multitude of health care visits and treatment periods in hospitals every year as well as non-attendance in day care centers and jobs. In most drastic cases, the respiratory infections may cause premature death of the elderly. However, the majority of respiratory tract infections are mild, self-limiting viral upper respiratory infections, also known as the common cold.

There are several clinical studies that have examined the effects of probiotics on respiratory infections in basically healthy children or adults. For example in school children Lactobacillus casei has reduced the occurrence of lower respiratory tract infections (Cobo Sanz J M. et al. 2006, Nutr Hosp 21, 547-51), while the same probiotic has reduced the duration of all infections in elderly subjects (Turchet P. et al. 2003, J Nutr Health Aging 7, 75-7). Furthermore, Lactobacillus rhamnosus (LGG) given in milk shows a relative reduction of 17% in the number of children suffering from respiratory infections with complications and lower respiratory tract infections (Hatakka K. et al. 2001, BMJ 322, 1-5), but in marathon runners LGG given in the form of milk based fruit drink or capsules did not have effect on respiratory infections (Kekkonen R. et al. 2007, Int J Sport Nutr Exerc Metab 17, 352-363). Also, a combination of LGG, Lactobacillus rhamnosus LC705 (LC705), Bifidobacterium breve Bb99 and Propionibacterium freudenreichii ssp shermanii has shown decrease of respiratory infections in children (Hatakka K. et al. 2007, Clin Nutr 26, 314-321; Kukkonen K. et al. 2008, Pediatrics 122, 8-12) but not in the elderly (Hatakka K. et al. 2007, doctoral thesis, http://urn.fi/URN:ISBN:978-952-10-3897-6).

In adults, for example dietary supplement containing Lactobacillus gasseri, Bifidobacterium longum and Bifidobacterium bifidum together with vitamins and minerals has shown reduced incidence of respiratory tract infections (Winkler P. et al. 2005, Int J Clin Pharmacol Ther 43, 318-26; de Vrese M. et al. 2005, Clin Nutr 24, 481-91).

Uncomplicated respiratory infections are widely treated by antibiotics. However, antibiotics do not have effect on viral infections. Therefore, the treatment of for example common cold is mainly based on symptom-relieving medications and fever reducing drugs. Some antiviral medication has been presented against influenza viruses, but no effective antiviral medication is so far available against other common respiratory viruses. Thus, effective drugs and treatments are still warranted for preventing or treating respiratory infections.

Also, it has been depicted in many previous studies that the effects of probiotics on respiratory infections differ between the age groups (e.g. infants, children, adults, the elderly). One bacterial strain having probiotic effects may be utilized in treating defined symptoms, but probiotics containing several strains of the same genera or different genera may provide further or different advantages for example by synergism or additive effects. Therefore, optimal probiotics or their combinations as well as preferred doses and consumption periods are warranted for different age groups suffering from respiratory infections.

### BRIEF DESCRIPTION OF THE INVENTION

The object of the present invention is to provide novel products, methods and uses for preventing or treating respiratory infections. Indeed, the present invention provides optimal probiotics or combinations thereof for these purposes. Positive effects of LC705 or LGG and LC705 have never before been detected on respiratory infections and furthermore, LGG has never before been shown to have effects on respiratory infections in adults.

The present invention relates to a composition comprising probiotics consisting of Lactobacillus rhamnosus LC705 alone or Lactobacillus rhamnosus GG and Lactobacillus rhamnosus LC705.

Furthermore, the present invention relates to a composition comprising probiotics consisting of Lactobacillus rhamnosus LC705 alone or Lactobacillus rhamnosus GG and Lactobacillus rhamnosus LC705 for use as a medicament.

Furthermore, the present invention relates to a use of Lactobacillus rhamnosus LC705 alone or together with Lactobacillus rhamnosus GG for the manufacture of a composition for the treatment and/or prevention of a respiratory infection in a subject.

Still, the present invention relates to a use of Lactobacillus rhamnosus LC705 alone or together with Lactobacillus rhamnosus GG for the manufacture of a composition for intensifying resistance against viruses causing respiratory infections in a subject.

Also, the present invention describes a use of Lactobacillus rhamnosus GG for the manufacture of a composition for the treatment and/or prevention of a respiratory infection in an adult subject.

The present invention also describes a use of Lactobacillus rhamnosus GG for the manufacture of a composition for intensifying resistance against viruses causing respiratory infections in an adult subject.

The present invention relates to a use of Lactobacillus rhamnosus for the manufacture of a composition for reducing, delaying or inhibiting influenza virus replication in a subject.

The present invention relates to a use of Lactobacillus rhamnosus for the manufacture of a composition for increasing an antiviral cytokine(s) in a subject to be or being infected with a respiratory infection.

Still, the present invention relates to Lactobacillus rhamnosus LC705 alone or together with Lactobacillus rhamnosus GG for the treatment and/or prevention of a respiratory infection in a subject.

The present invention relates to Lactobacillus rhamnosus LC705 alone or together with Lactobacillus rhamnosus GG for intensifying resistance against viruses causing respiratory infections in a subject.

Also, the present invention describes Lactobacillus rhamnosus GG for the treatment and/or prevention of a respiratory infection in an adult subject.

The present invention also describes Lactobacillus rhamnosus GG for intensifying resistance against viruses causing respiratory infections in an adult subject.

The present invention relates to Lactobacillus rhamnosus for reducing, delaying or inhibiting influenza virus replication in a subject.

The present invention relates to Lactobacillus rhamnosus for increasing an antiviral cytokine(s) in a subject to be or being infected with a respiratory infection.

Also, the present invention relates to a method of treating or preventing a respiratory infection in a subject, wherein the method comprises administration of a composition comprising Lactobacillus rhamnosus LC705 alone or together with Lactobacillus rhamnosus GG to the subject.

The present invention also describes a method of treating or preventing respiratory infection in an adult subject, wherein the method comprises administration of a composition comprising Lactobacillus rhamnosus GG to the adult subject.

Still the present invention relates to a method of intensifying resistance against viruses causing respiratory infections in a subject, wherein the method comprises administration of a composition comprising Lactobacillus rhamnosus LC705 alone or together with Lactobacillus rhamnosus GG to the subject.

Still the present invention relates to a method of reducing, delaying or inhibiting influenza virus replication in a subject, wherein the method comprises administration of a composition comprising Lactobacillus rhamnosus to the subject.

Still the present invention relates to a method of increasing an antiviral cytokine(s) in a subject to be or being infected with a respiratory infection, wherein the method comprises administration of a composition comprising Lactobacillus rhamnosus to the subject.

The present invention provides tools for further developments in food, feed and pharmaceutical industries. Furthermore, by the present invention more effective and specific treatments become available to patients, e.g. to different age groups, suffering from respiratory infections.

LGG, LC705 or a combination thereof can be used as such or as a part of another product, such as a pharmaceutical or a food product. LGG, LC705 or a combination of the invention has an advantageous antiviral effect on a human being by increasing the expression of antiviral proteins (e.g. IP-10 and IFN-α (FIG. 1), Mx1, Mx2 and RIG-I (FIG. 2)) and by preventing the proliferation of influenza viruses, which can be seen for example by decrease of the viral structural proteins (e.g. NP and M1 (FIG. 5)).

There is a continued, evident need to offer the consumers new products having clearly demonstrated effects on health, specifically on respiratory infections, and produced in a form that allows them to be easily used as such or as a part of another product, such as a pharmaceutical or a food or feed product.

### BRIEF DESCRIPTION OF THE FIGURES

FIG. 1 shows that LC705 induces antiviral IFN-α production in macrophages obtained from human monocytes. 6 hours and 24 hours stimulations of macrophages with LGG or LC705 are shown in the figure. LGG or LC705 induces antiviral IP-10 production but LC705 induces IP-10 production more than LGG.

FIG. 2 shows that LGG or LC705 activates Mx1, Mx2 and RIG-I production. However, LC705 activates IFN-α regulated antiviral protein (Mx1, Mx2, RIG-I) production more than LGG.

FIG. 3 shows that LGG, LC705 and a combination thereof increase IL-1β (FIG. 3A), LC705 increases IFN-α (FIG. 3B), and LC705 and a combination of LGG and LC705 increase TN F-α (FIG. 3C) production. Furthermore, FIG. 3 shows that LC705 and combination of LGG and LC705 increase anti-viral inflammatory activity (IL-1β and TNF-α (FIGS. 3A and 3C)) during influenza A virus infection more than LGG. LC705 and combination of LGG and LC705 increase antiviral IFN-α production during influenza A virus infection more than LGG (FIG. 3B).

FIG. 4 shows that LGG, LC705 or a combination thereof decrease synthesis of influenza A virus mRNA, i.e. decrease or slow down replication of the virus. FIG. 4A shows that LC705 or a combination of LGG and LC705 decreases or slows down production of NP mRNA during virus infection. FIG. 4B shows that LGG, LC705 or a combination thereof decreases or slows down production of M1 mRNA during virus infection. FIG. 4C shows that LGG, LC705 or a combination thereof decreases or slows down production of NS1 mRNA during virus infection.

FIG. 5 shows that LGG or LC705 decreases the production of structural proteins (NP, M1) of influenza A virus.

### DETAILED DESCRIPTION OF THE INVENTION

Probiotics have been utilized in food and feed industry for a long time, but still, effects of probiotics on wide-ranging symptoms or diseases and on specific target groups need to be determined. The present invention resides in findings that LGG and LC705 have probiotic effects on respiratory infections. Also, LC705 and LGG function as an optimal combination for intensifying production of antiviral cytokines and preventing the amplification of viruses, thus reducing the risk of respiratory infections.

### Probiotic Bacteria

Probiotics are live micro-organisms, preferably non-pathogenic microbes which, when administered in adequate amounts to man or animal, promote the well being of the host (Fuller R 1989, J Appl Microbiol 66, 365-378). Probiotics will result in a beneficial health advantage to the host, when consumed as a food or a food supplement in adequate amounts.

Health claims of probiotics in humans or animals include the possible prevention and treatment of many ailments. The health-promoting effects of probiotics include for example the balancing and maintenance of intestinal flora, stimulation of the immune system and anti-carcinogenic activity.

The best-documented probiotics include L. rhamnosus GG, L. johnsonii LA1, L. casei Shirota and Bifidobacterium lactis Bb12. In addition, a number of other probiotics, such as L. rhamnosus LC705 have been described in the literature.

Lactobacillus rhamnosus GG (LGG, LGG®) strain is a non-pathogenic Gram-positive isolate originally from the USA (U.S. Pat. No. 4,839,281 A). LGG strain is isolated from human feces, it is able to grow well in pH 3 and survives even lower pH values as well as high bile acid contents. The strain exhibits excellent adhesion to both mucus and epithelial cells, and colonizes GIT. Lactic acid yield from glucose is good: when grown in MRS broth, the strain produces 1.5-2% of lactic acid. The strain does not ferment lactose and thus it does not produce lactic acid from lactose. The strain ferments following carbohydrates: D-arabinose, ribose, galactose, D-glucose, D-fructose, D-mannose, rhamnose, dulcitol, inositol, mannitol, sorbitol, N-acetylglucosamine, amygdalin, arbutin, esculin, salicin, cellobiose, maltose, saccharose, trehalose, melezitose, gentibiose, D-tagatose, L-fucose, and gluconate. The strain grows well at 15-45° C., the optimum temperature being 30-37° C. LGG has been deposited with the depository authority American Type Culture Collection under accession number ATCC 53103.

Lactobacillus rhamnosus LC705 (LC705) strain is also a non-pathogenic Gram-positive isolate, but originally from Finland. LC705 is described in greater detail in Fl Patent 92498, Valio Oy. LC705 is a gram-positive short rod occurring in chains; it is homofermentative; weakly proteolytic; grows well at +15-45° C.; does not produce ammonia from arginine; is catalase-negative; when grown in MRS broth (LAB M), the strain produces 1.6% lactic acid having an optical activity of the L(+) configuration; the strain decomposes citrate (0.169%), thereby producing diacetyl and acetoin; the strain ferments at least the following carbohydrates (sugars, sugar alcohols): ribose, galactose, D-glucose, D-fructose, D-mannose, L-sorbose, rhamnose, mannitol, sorbitol, methyl-D-glucoside, N-acetylglucosamine, amygdalin, arbutin, esculin, salicin, cellobiose, maltose, lactose, sucrose, trehalose, melezitose, gentiobiose, D-turanose and D-tagatose. LC705 adheres weakly to mucus cells, but moderately to epithelial cells. The viability of the strain is good in low pH values and high bile acid contents. The strain survives well a salinity of 5% and fairly well a salinity of 10%. LC705 is deposited with the Deutsche Sammlung von Mikro-organismen and Zellkulturen GmbH (DSM) under accession number DSM 7061.

In one embodiment of the invention, Lactobacillus rhamnosus is LGG. In another embodiment of the invention, Lactobacillus rhamnosus is LC705. In a specific embodiment of the invention, Lactobacillus rhamnosus is LGG and LC705.

### Compositions

The composition of the present invention comprises LGG and LC705 probiotics. Only probiotics LGG and LC705 are comprised in the composition. Compositions of the invention may be selected from, but are not limited to, the group consisting of food products, animal feed, nutritional products, food supplements, food ingredients, health food, pharmaceutical products and cosmetics. Compositions are also applicable as convenient part or supplement, for example, of the every-day diet or medication. In one preferred embodiment of the invention, the composition is a pharmaceutical, food or feed product. In another embodiment of the invention the composition is functional food, i.e.

food having any health promoting and/or disease preventing or treating properties. Preferably a food product of the invention is selected from the group consisting of dairy products, bakery product, chocolate and confectionary, sugar and gum confectionary, cereal products, snacks, berry or fruit based products and drinks/beverages. Dairy products include but are not limited to milk, sour milk, yogurts and other fermented milk products such as cheeses and spreads, milk powders, children's food, baby food, toddler's food, infant formula, juices and soups.

The composition of the invention may be a pharmaceutical composition and may be used for example in solid, semisolid or liquid form such as in the form of tablets, pills, pellets, capsules, solutions, emulsions or suspensions. Preferably the composition is for oral administration or for enteral, inhalable or intravenous applications. Furthermore, it may be administered to a subject before or after the subject has been infected with a respiratory infection.

In addition to probiotics, the composition may comprise pharmaceutically or nutritionally acceptable and/or technologically needed carrier(s) (e.g. water, glucose or lactose), adjuvant(s), excipient(s), auxiliary excipient(s), antiseptic(s), stabilizing, thickening or coloring agent(s), perfume(s), binding agent(s), filling agent(s), lubricating agent(s), suspending agent(s), sweetener(s), flavoring agent(s), gelatinizer(s), anti-oxidant(s), preservative(s), buffer(s), pH regulator(s), wetting agent(s), starter(s) or components normally found in corresponding compositions. Any agent, which is not a probiotic may for example be selected from the above-mentioned group. Agents of a composition, e.g. ingredients or components, are either obtained commercially or prepared by conventional techniques known in the art.

In a specific embodiment of the invention, the composition comprises probiotic agents consisting of LGG and LC705, and optionally any non-probiotic agents. “Non-probiotic agent” refers to any agent, which is not a probiotic.

The composition of the invention comprises LGG and/or LC705 in an amount sufficient to produce the desired effect. In a preferred embodiment of the invention, the proportions (bacterial numbers) of LGG and LC705 are equal i.e. 1:1. In another preferred embodiment of the invention, the proportion (bacterial numbers) of LGG to LC705 is 2:1, 3:1, 4:1, 5:1, 6:1, 7:1, 8:1, 9:1 or 10:1. In another preferred embodiment of the invention, the proportion (bacterial numbers) of LC705 to LGG is 2:1, 3:1, 4:1, 5:1, 6:1, 7:1, 8:1, 9:1 or 10:1.

In addition to Lactobacillus rhamnosus probiotics, a composition used for treating and/or preventing a respiratory infection, for intensifying resistance against viruses causing respiratory infections, for reducing, delaying or inhibiting influenza virus replication, or for increasing an antiviral cytokine(s) may also comprise other probiotics or any other agents normally found in corresponding compositions.

The compositions may be manufactured by any conventional processes known in the art. LGG and/or LC705 may for example be added to any products or mixed with any agents either in connection with the preparation or thereafter, during the finishing of the end product.

### Respiratory Infections and Treatments

Respiratory infections include infections of both the upper and lower respiratory tract. Upper respiratory tract infection involves inflammation of the respiratory mucosa from the nose to the lower respiratory tree, excluding alveoli. Thus, upper respiratory tract includes nasal cavity (nose, sinuses), pharynx and larynx. Upper respiratory tract infections are selected from, but are not limited to, the group consisting of common cold, sinusitis, ear infection, otitis, mastoiditis, pharyngitis, tonsillitis, epiglottitis, tracheitis, laryngitis and bronchitis. Symptoms of upper respiratory tract infections include nasal congestion, cough, rhinitis, blocked nose, running nose, sore throat, fever, facial pressure, headache, loss of apetite and/or sneezing.

Lower respiratory tract infections involve trachea, primary bronchi and lungs. Infections affecting the lower respiratory tract may be selected from, but are not limited to, the group consisting of pneumonia, pleuritis, bronchitis, bronchiolitis, and emphysema, and symptoms include for example shortness of breath, weakness, high fever, coughing and/or fatigue.

A large number of bacterial species colonise the upper respiratory tract, while the lower respiratory tract is normally virtually free of microorganisms. Symptoms of the upper or lower respiratory tract infections arise after exposure to a pathogen and an incubation period ranging from hours to days (e.g. 1-7 days), and may last for example from three to ten days or even longer (weeks). The nature and duration of the symptoms depends on the pathogen, amount of pathogens as well as the age and immunological condition of a subject.

In addition to acute infections, pathogens may also cause chronic infections. A chronic infection develops usually from an acute infection and can last for days to a lifetime.

As used herein “infection” refers to an invasion and multiplication of pathogenic microorganisms in a cell or tissue, i.e. “infection” also refers to a state resulting from having been infected. Infection may cause injury and progress to a disease through a variety of cellular or toxic mechanisms. However, all infections do not lead to clinical illness; symptomatic diseases are known to develop in 75% of infected persons (Gwaltney J M and Hayden F G, 1992, N Engl J Med 326, 644-5).

Pathogens causing respiratory infections may be bacteria or viruses. In some cases, the infections are a consequence of both of them. Bacteria or viruses causing respiratory infections i.e. upper and/or lower respiratory tract infections may be selected from the group consisting of Haemophilus influenzae, Streptococcus pneumoniae, Moraxella catarrhalis, Streptococcus pyogenes, Staphylococcus aureus, Mycoplasma pneumoniae, Chlamydiae pneumoniae, common cold (influenza) virus, rhinovirus, adenovirus, parainfluenza virus, respiratory syncytial virus, enterovirus, coronavirus and Epstein-Barr virus. Also, any other bacteria or virus affecting respiratory tract may be included to the above-mentioned group. Viruses causing respiratory infections, which can be prevented or treated with the compositions of the invention, may be selected from the group consisting of, but not limited to, common cold (influenza) virus, rhinovirus, adenovirus, parainfluenza virus, respiratory syncytial virus, enterovirus, coronavirus and Epstein-Barr virus.

In one embodiment of the invention, the respiratory infection is an influenza virus infection. In a preferred embodiment of the invention, the influenza virus is selected from the group consisting of influenza virus A and influenza virus B. Influenza viruses A and B belong to a group of single stranded RNA viruses and to a family of Orthomyxoviridae viruses. Influenza viruses A are hosted by birds and cause “avian influenza”, which is also known as a bird flu or avian flu. All known subtypes of the virus are endemic in birds. However, influenza virus A may also infect mammals, and at least subtypes named H1N1, H2N2, H3N2 and H5N1 have been detected in humans. Influenza viruses B are only known to infect humans and seals, and, in contrast to influenza virus A, influenza virus B does not cause influenza pandemics.

Virus particles do not grow or amplify by themselves and they also lack genetic information for protein synthesis and energy production. That is why they are dependent on the host cells. The pathogenic mechanisms of various respiratory viruses differ between the viruses. The understanding of the pathogenetic events is mainly derived from rhinovirus infections. Rhinoviruses are transmitted mainly by small aerosol particles, and via direct or indirect contact with infected secretions. At the beginning of the infection, the rhinovirus invades the host by binding to the ICAM-1 receptor (intercellular adhesion receptor molecule 1), mainly located in the nasopharynx. After intracellular invasion and replication, the virus spreads intranasally to the pharynx. Replication evokes inflammatory and immune responses in the host, leading to vasodilatation, increased vascular permeability and cellular infiltration, through the release of inflammatory mediators. Elevated concentrations of proinflammatory cytokines result in a cascade of inflammatory reaction necessary to eradicate or neutralize the virus (van Kempen M. et al. 1999, Rhinology 37, 97-103).

The influenza viruses infect host epithelial cells by binding to receptors on the cell surface via one of the major viral surface glycoproteins, the hemagglutinin (HA). The host respiratory tract is not only the site of infection for influenza viruses, but also the site of defence against viral infection. Defence mechanisms against influenza virus infection comprise several effector cells and molecules. Viruses are initially detected and destroyed non-specifically by innate immune mechanisms, which are not antigen specific and do not require a prolonged period of induction. Several components such as mucus, macrophages, dendritic cell (DCs) natural killer (NK) cells, interferon (IFN) α, β and other cytokines, and complement components are involved in the innate immune system. The presence of the viruses in an epithelial cell induces IFN-α and IFN-β production. Furthermore, the cytokines such as IL-1, IL-6, TNF-α and IL-12 secreted by the macrophages activate NK cells. NK cells release IFN-γ, which among others affects the lysis of the infected cells. Binding of interferon to the cell surface receptors increases the transcription of many genes, which furthermore accelerates for example the production of cytokines. Interferons may also activate ribonuclease enzyme, which degrades viral RNA, and moreover, interferons may interrupt protein synthesis indirectly for inhibiting viral replication (Tamura S. and Kurata T., 2004, Jpn J Infect Dis 57:236-247; Tamura S. et al. 2005, Jpn J Infect Dis, 58:195-207).

However, if the viruses avoid the early defence mechanisms, they are detected and eliminated specifically by the adaptive immune mechanisms, which could be augmented by influenza virus constituents via Toll-like receptors (TLRs) on macrophages and DCs in the respiratory tract. Macrophages and DCs, which have recognised viruses, present viral antigens to T- and B-lymphocytes with the aid of MHC-1 (HLA-I and HLA-II in humans) and MHC-2 proteins. This series of events starts the adaptive immune response. The production of antibodies is activated in order to neutralize the viruses (Tamura S. and Kurata T., 2004, Jpn J Infect Dis 57:236-247; Tamura S. et al. 2005, Jpn J Infect Dis, 58:195-207).

Alterations of an immune response can be monitored by any suitable medical, physiological or biological test e.g. in vitro, ex vivo or in vivo test from any biological sample or subject. The properties of probiotic strains may be investigated for example in cell cultures (in vitro) utilizing for example peripheral blood mononuclear cells (PBMC), human monocytes, macrophages and dendrite cells. Examples of ex vivo experiments include determination of phagocytosis of neutrophils and monocytes, oxidative burst i.e. superoxide generation of neutrophils and monocytes, natural killer (NK) cell activity, lymphocyte proliferation and production of cytokines by PBMC, tissue macrophages, monocytes or lymphocytes. In vivo experiments include but are not limited to determination of a response to vaccines (e.g. vaccine specific antibodies or vaccine-specific antibody forming cells), delayed type hypersensitivity and response to attenuated pathogens.

The major cells protecting the host against the invasion of pathogens are macrophages, which eat i.e. phagocytose the pathogens or produce cytokines. Cytokines recruit other immune cells and mediate inflammation. Macrophages are white blood cells within tissues and can be cultured in vitro by the differentiation of monocytes. In the present invention, macrophages from healthy adults were used in an in vitro model for studying the effects of probiotics on human subjects. Macrophages were stimulated with LGG and/or LC705 and infected with influenza viruses.

To activate immune responses, macrophages produce cytokines, chemokines and antimicrobial substances. Cytokines are signaling molecules (i.e. proteins, peptides, or glycoproteins) that are used in cellular communication.

They are often secreted by immune cells that have encountered a pathogen, thereby activating and recruiting further immune cells to increase the system's response to the pathogen.

Each cytokine has a matching cell-surface receptor and thus, subsequent cascades of intracellular signaling alter cell functions. Intracellular signaling may lead for example to the upregulation and/or downregulation of several genes and their transcription factors, resulting in the production of other cytokines, an increase in the number of surface receptors for other molecules, or the suppression of their own effect by feedback inhibition.

Cytokines can be divided into two groups: type 1, those enhancing cytokine responses (eg. IFN-γ, TGF-β) and type 2, favoring antibody responses (eg. IL-4, IL-10, IL-13). Proinflammatory cytokines tumor necrosis factor alpha (TNF-α), interleukin-1β (IL-1β) and IL-6 as well as interferons (IFNs) are among the first cytokines produced in response to microbial infection. Cytokines produced later during microbial infection direct responses toward either cell-mediated T-helper type 1 (Th1) or humoral Th2 type immunity.

Chemokines are a family of small cytokines (approximately 8-10 kilodaltons in size and four cysteine residues in conserved locations) which induce directed chemotaxis of nearby responsive cells. Some chemokines such as IP-10 are considered inflammatory. These proteins exert their biological effects by interacting with G protein-linked transmembrane receptors.

Tests for detecting alterations of an immune response include but are not limited to those that are based on detecting activation of signalling pathways as well as detecting a transcription or translation level of marker genes or the amount of proteins (e.g. antibodies or receptors). A single marker is not currently available for determining the immune response in a cell or organism. However, preferable markers may be selected from the group consisting of, but not limited to, TNF-α, IL-12, IL-10, IL-1β, IFN-α, IL-1α, IL-6, IL-18, IFN-γ, IL-4, TGF-β, and IP-10.

Probiotic stimulation is known to induce production of IL-1β, IL-6 and TNF-α in macrophages (Miettinen M. et al. 2008, J Leukoc Biol. 84: 1092-1100), but LGG or LC705 induced effects on influenza virus infections have never been shown in macrophages.

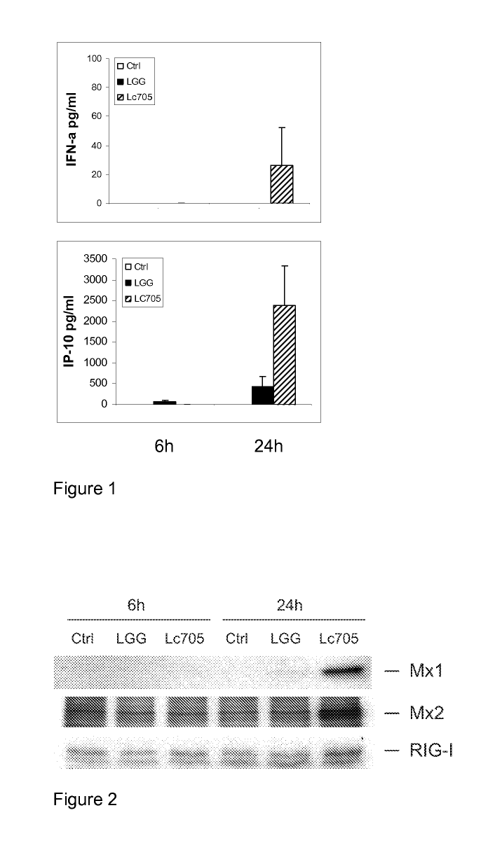

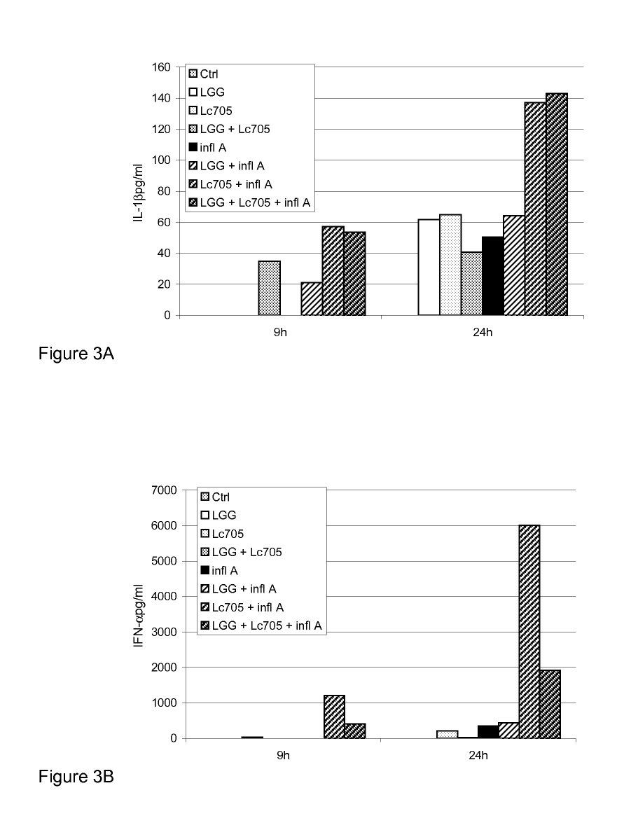

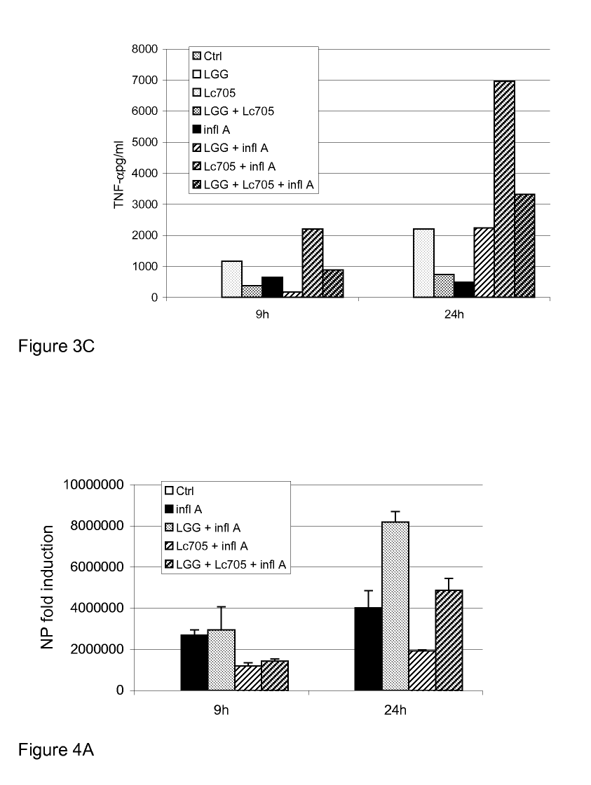

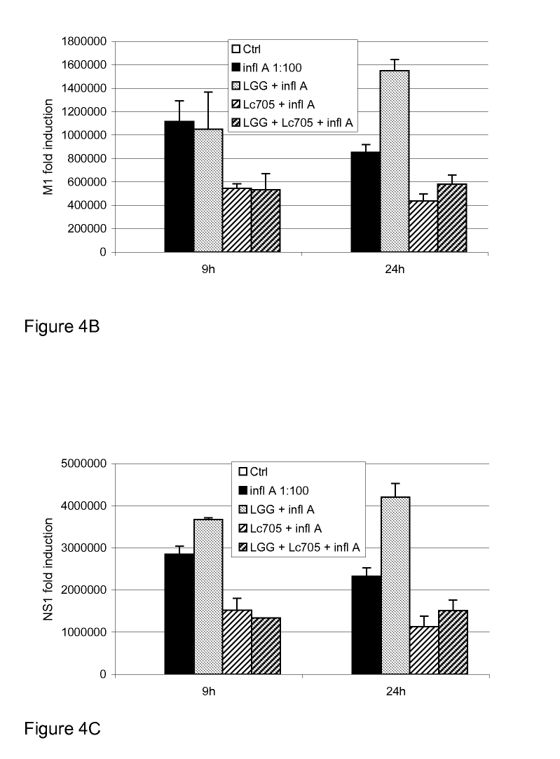

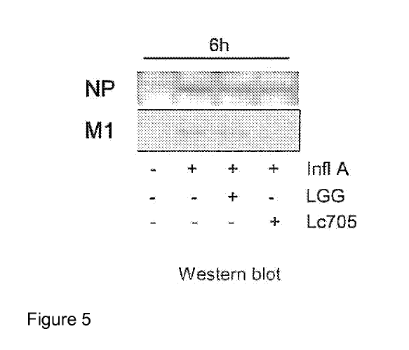

In the present invention, LGG and/or LC705 increased antiviral proteins (IP-10, TNF-α, IL-1β, IFN-α and IFN-β (FIGS. 1 and 3)) and IFN-α inducible cytokines or proteins (Mx1, Mx2, and RIG-I (FIG. 2)), and thus, slowed down the function of viruses. The slowed down function of viruses was also detected by decreased amount of viral structural proteins (FIGS. 4 and 5).

In the present invention, LGG and/or LC705 increase the antiviral cytokine production and thus participate in inactivating the viruses. This phenomenon is also referred to as “intensifying the resistance against viruses”. “Antiviral cytokines” refers to cytokines that help in destroying or neutralizing the viruses. In a preferred embodiment of the invention, antiviral cytokine(s) is/are selected from the group consisting of IFN-α, IFN-β, IL-1β, TNF-α and IP10. Type 1 interferons (IFN-α/β) are essential for example in debating against influenza virus infections. IFN-α inducible cytokines or proteins include but are not limited to Mx1, Mx2, RIG-1 and IP-10.

Mx1 protein is a Myxovirus (influenza virus) resistance 1 protein ((interferon-inducible protein p78 (mouse)), which is also known as MxA in humans. Cytoplasmic protein Mx1 is a member of both the dynamin family and the family of large GTPases. Interferon-inducible Mx1 protein shows activity against influenza virus by interfering with the role of virus nucleocapsid (NP) in viral replication.

Expression of Mx gene is mainly regulated by type I IFNs. Signalling pathways from IFNs induce activation of IFN-stimulated response element (ISRE) upstream of Mx gene (Hug H. et al. 1988. Mol Cell Biol 8, 3065-3079).

Also virus infection or administration of double-stranded RNA (dsRNA) per se can produce a quick and efficient Mx gene activation (Hug H. et al. 1988. Mol Cell Biol 8, 3065-3079; Ronni T. et al. 1995. J Immunol 154, 2764-2774). In all cases Mx induction is a true primary response to the virus, rather than a secondary response to virus-induced IFN. Cells are capable of reacting rapidly on infection by simultaneously synthesizing Mx protein that will remain intracellular and IFNs that will be released into the cellular environment. This interferon induces expression of Mx protein in neighboring uninfected cells, such that the cells initially infected soon become demarcated by a barrier of virus-protected cells. Consequently, virus can not spread efficiently, giving the immune system enough time to mount its own line of defense and eliminate the virus.

Cytoplasmic protein Mx2 (Myxovirus (influenza virus) resistance 2)), known as MxB in humans, is a member of both the dynamin family and the family of large GTPases. The protein has also a nuclear form, which is localized in a granular pattern in the heterochromatin region beneath the nuclear envelope. A nuclear localization signal (NLS) is present at the amino terminal end of the nuclear form. This protein is upregulated by IFN-α.

RIG-1 (DDX58, Retinoic acid-inducible gene 1 protein, DEAD-box protein 58) is an IFN-α inducible RNA helicase. RIG-1 contains 2 CARD domains, a helicase ATP-binding domain and a helicase C-terminal domain. RIG-1 has an essential function in double stranded RNA-induced innate antiviral responses in preventing viral replication. RIG-1 is also known to be activated by single stranded RNA in the case of influenza A virus infection. Influenza A virus NS1 protein binds RIG-I which prevents the antiviral actions of RIG-I (Pichlmair A. et al. 2006, Science, 314 (5801):997-1001).

Interferon (IFN)-inducible protein 10 (IP-10) is a member of the chemokine family of cytokines and is induced in a variety of cells in response to IFN-γ, IFN-α and lipopolysaccharide. IP-10 binding sites have been detected on a variety of cells including endothelial, epithelial, and hematopoietic cells. IP-10 gene expression has been shown to be elevated by influenza A viruses.

In the methods or uses of the invention, Lactobacillus rhamnosus is administered to a subject for reducing, delaying or inhibiting influenza virus replication. Replication efficiency of viruses can be studied by determining the amount of viral mRNA or structural proteins. LGG and/or LC705 reduce, delay or inhibit the influenza virus replication, and therefore, reduce or prevent the symptoms normally caused by the infection. Suitable influenza virus proteins for determining the viral replication efficiency may for example include, but are not limited to, NP (nucleoprotein), NS1 (nonstructured protein 1), polymerase proteins PB1, PB2 or PA, external glycoproteins HA (hemagglutinin) or NA (neuraminidase), M1 (matrix protein), M2 or NS2. In the present invention, the effect of LGG and/or LC705 on mRNAs or proteins of NP, NS1, and M1 was studied and LGG and/or LC705 decreased or slowed down the production of all of them.

NS1 is a non-structural protein 1, which hinders the transport of the host mRNA from a nucleus, hampers RIG-I mediated IFN-response, and inhibits antiviral condition. NP, for one, is a nucleoprotein, which is connected to every gene fragment and regulates transport to the nucleus. M1 is a matrix protein, which is located in the inner matrix of the viral lipid envelope. Accumulation of M1 is required for virus budding.

In the present invention, a subject for treatments or preventions can be any eukaryotic organism, preferably a human being. In a preferred embodiment of the invention, the subject is an infant, child or adult. “Infant” refers to a person with age of 0 to 5 months, “child” refers to a person with age of 6 months to 17 years and “adult” refers to a person with age of 18 years or more. The subject may also be an animal, especially a pet or a production animal. The animal may be selected from the group consisting of production animals and pets, such as cows, horses, pigs, goats, sheep, poultry, dogs, cats, rabbits, reptiles and snakes.

In the present invention, Lactobacillus rhamnosus or compositions comprising Lactobacillus rhamnosus (e.g. LGG and/or LC705) may be administered to a subject either before or after the subject has been infected with a respiratory infection.

In specific embodiments of the invention, LGG and LC705 are used for the treatment and/or prevention of a respiratory infection in a subject, for intensifying resistance against viruses causing respiratory infections in a subject, for reducing, delaying or inhibiting influenza virus replication in a subject or for increasing an antiviral cytokine(s) in a subject to be or being infected with a respiratory infection.

The present invention is illustrated by the following examples, which are not intended to be limiting in any way.

### EXAMPLE 1

### Macrophages and Probiotic Bacteria

### Macrophages

Freshly collected, leukocyte-rich buffy coats from healthy blood donors were supplied by the Finnish Red Cross Blood Transfusion Service. PBMCs were isolated by a density gradient centrifugation. Monocytes were purified from PBMCs by adherence on six-well plastic plates (Falcon) and cultured for 7 days in macrophage-serum-free medium (Gibco Invitrogen) in the presence of recombinant human (rh)GM-CSF (Leucomax, Schering-Plough) to obtain macrophages as described previously (Miettinen M. et al. 2000, J Immunol 164, 3733-3740).

### Bacteria

Lactobacillus rhamnosus GG and Lactobacillus rhamnosus LC705 were stored in skimmed milk at −70° C. and passaged three times as previously described (Miettinen M. et al. 1996, Infect Immun 64:5403) before their use in stimulation experiments. Lactobacilli were grown in MRS medium (Difco). For stimulation experiments the bacteria were grown to logarithmic growth phase, and the number of bacterial cells was determined by counting in a Petroff Hausser counting chamber.

### EXAMPLE 2

### Stimulation Experiments on Macrophages

Stimulation experiments were conducted in RPMI 1640 medium (Sigma). The macrophages of example 1 were stimulated for 24 hours with live bacteria of example 1, i.e. with LGG or LC705 alone at a 1:1 ratio (by cell number) or with LGG and LC705 together at an equal bacterial cell number with bacteria to macrophage cell ratio remaining 1:1. Macrophages were then infected with multiplicity of infection (MOI) 5 of influenza A/Beijing/353/89 virus (0.128 HAU/ml) (Institute for Health and Welfare (THL)) for 1 hour leading to 100% infection, after which the infected cells were washed with PBS, cell culture medium changed, and infection continued for a total of 9 or 24 hours as described earlier (Pirhonen J. et al. 1999. J Immunol 162, 7322-7329). The cells and cell culture supernatants were collected after stimulations. Total RNA or protein was isolated from collected samples. The amount of viral mRNA was determined by quantitative real-time PCR qRT-PCR) and virus or host proteins were detected by Western blot. Secreted cytokines were measured by ELISA method.

MxA, MxB, RIG-I, NP and M1 protein expression was analysed by Western blot method as previously described (Miettinen M. et al. 2008, J Leukoc Biol 84, 1092-1100) (see FIGS. 2 and 5) with the following antibodies: anti-MxA (Ronni T. et al. 1993. J immunol 150, 1715-1726; anti-MxB (Melén K. et al. 1996. J Biol Chem 271, 23478-23486); anti-RIG-I (Matikainen S. et al.

2006. J Virol 80, 3515-3522); influenza A-specific anti-NP (Ronni T. et al. 1995. J Immunol 154, 2764-2774); and influenza A specific anti-M1 antibody that was obtained by immunizing rabbits similarly as described for obtaining anti-NP (Ronni T. et al. 1995. J Immunol 154, 2764-2774).

Quantitation of viral mRNAs by qRT-PCR was performed with Applied Biosystems reagents and protocols as described (Miettinen M. et al. 2008, J Leukoc Biol 84: 1092-1100) with primer-probe pairs for influenza A M1 (Ward C L. et al. 2004. J Clin Virol 29, 179-188). NP primer-probes were following: forward primer 5′-ccataaggaccaggagtgga-3′, reverse primer 5′ccctccgtatttccagtgaa-3′, probe 5′-caggccaaatcagtgtgcaacctac-3′, and NS1 primer-probes were: forward primer 5′-tgaaagcgaatttcagtgtgat-3′, reverse primer 5′-ctggaaaagaaggcaatggt-3′, probe 5′-ctaagggctttcaccgaagaggg-3′.

Cytokine (IL-1β, IFN-α, TNF-α) and chemokine levels (IP-10) in cell culture supernatants were determined by ELISA methods as described previously (Miettinen M. et al. 1998, Infect Immun 66, 6058-6062; Veckman V. et al. 2003. J Leukoc Biol 74, 395-402 (see FIGS. 1 and 3-4).

1. A use of Lactobacillus rhamnosus LC705 alone or together with Lactobacillus rhamnosus GG for the preparation of a composition having an antiviral effect in a subject.

2. The use according to claim 1, wherein the composition is a pharmaceutical, food or feed product.

3. The use according to claim 2, wherein the composition is a pharmaceutical composition.

4. The use of claim 1, wherein the composition is for the treatment and/or prevention of a respiratory infection in a subject.

5. A method for treating and/or preventing a respiratory infection in a subject, wherein the method comprises administration of a composition comprising Lactobacillus rhamnosus LC705 alone or together with Lactobacillus rhamnosus GG to the subject, the composition having an antiviral effect in the subject.

6. The method of claim 5, wherein the composition is for intensifying resistance against viruses causing respiratory infections in a subject.

7. The method of claim 5, wherein the composition increases the expression of antiviral proteins.

8. The method according to claim 5, wherein the composition is for increasing an antiviral cytokine(s) in a subject to be or being infected with a respiratory infection.

9. The method according to claim 8, wherein the antiviral cytokine is selected from a group consisting of IFN-α, IFN-β, IL-1β, TNF-α, and IP-10.

10. The method of claim 8, wherein the respiratory infection is caused by a virus, which is selected from the group consisting of common cold (influenza) virus, rhinovirus, adenovirus, parainfluenza virus, respiratory syncytial virus, enterovirus, coronavirus, and Epstein-Barr virus.

11. The method according to claim 10, wherein the respiratory infection is an influenza virus infection.

12. The method according to claim 11, wherein the influenza virus is selected from the group consisting of influenza virus A and influenza virus B.

13. The method according to claim 10, wherein the composition is for preventing the proliferation of influenza virus and/or for reducing, delaying or inhibiting influenza virus replication in a subject.

14. Method according to claim 5, wherein the subject is an infant, child or adult.

15. An antiviral composition comprising Lactobacillus rhamnosus LC705 as a probiotic.

16. An antiviral composition comprising probiotics consisting of Lactobacillus rhamnosus LC705 and Lactobacillus rhamnosus GG.

17. Lactobacillus rhamnosus LC705 alone or together with Lactobacillus rhamnosus GG for treating and/or preventing a respiratory infection in a subject.

18. Lactobacillus rhamnosus LC705 alone or together with Lactobacillus rhamnosus GG for intensifying resistance against viruses causing respiratory infections in a subject.

19. Lactobacillus rhamnosus LC705 alone or together with Lactobacillus rhamnosus GG for increasing the expression of antiviral proteins.

20. Lactobacillus rhamnosus LC705 alone or together with Lactobacillus rhamnosus GG for increasing an antiviral cytokine(s) in a subject to be or being infected with a respiratory infection.

21. Lactobacillus rhamnosus LC705 alone or together with Lactobacillus rhamnosus GG for preventing the proliferation of influenza virus and/or for reducing, delaying or inhibiting influenza virus replication in a subject.


SEQUENCE LISTING (6 sequences)

SEQ ID NO: 1 - DNA, 20 bp, Artificial
  oligonucleotide
ccataaggaccaggagtgga

SEQ ID NO: 2 - DNA, 20 bp, Artificial
  oligonucleotide
ccctccgtatttccagtgaa

SEQ ID NO: 3 - DNA, 25 bp, Artificial
  oligonucleotide
caggccaaatcagtgtgcaacctac

SEQ ID NO: 4 - DNA, 22 bp, Artificial
  oligonucleotide
tgaaagcgaatttcagtgtgat

SEQ ID NO: 5 - DNA, 20 bp, Artificial
  oligonucleotide
ctggaaaagaaggcaatggt

SEQ ID NO: 6 - DNA, 23 bp, Artificial
  oligonucleotide
ctaagggctttcaccgaagaggg